(19) United States
(12) Patent Application Publication
(10) Pub. No.: US 2022/0229819 A1
(43) Pub. Date: Jul. 21, 2022

(54) METHODS, MEDIUMS, AND SYSTEMS FOR CONFIGURING A DNA/RNA TARGET PROBE DESIGN

(71) Applicant: Applied Materials, Inc., Santa Clara, CA (US)

(72) Inventor: Bongjun Son, San Jose, CA (US)

(73) Assignee: Applied Materials, Inc., Santa Clara, CA (US)

(21) Appl. No.: 17/376,582

(22) Filed: Jul. 15, 2021

Related U.S. Application Data
(60) Provisional application No. 63/140,086, filed on Jan. 21, 2021.

(51) Int. Cl. G06F 16/21; G06F 16/245; G16H 70/60; G16H 50/70; G16H 40/20; G16B 25/20; G16B 50/30

(52) U.S. Cl. CPC G06F 16/211; G06F 16/245; G16H 70/60; G16B 50/30; G16H 40/20; G16B 25/20; G16H 50/70

(57) ABSTRACT

Exemplary embodiments provide methods, mediums, and systems for generating a library of oligonucleotides for fluorescence in-situ hybridization transcriptomics probes. The illustrative techniques include several improvements, which may be utilized separately or together. These improvements include automatically iterating over a particular group of probe building actions while excluding other actions from the automatic iterations. This serves to reduce the amount of processing and memory resources required while significantly speeding up the process of building the library. Other improvements described simplify the input of genes of interest to be used to construct the probes and provide quality control capabilities. The described solution may be implemented in non-script-based instructions, which simplifies the input procedure, allows for the separation of data management and processing capabilities, and reduces the need for expert users to build the library.

### CROSS-REFERENCE TO RELATED APPLICATION

The present application claims priority to U.S. Provisional patent application No. 63/140,086, filed Jan. 21, 2021, entitled “METHODS, MEDIUMS, AND SYSTEMS FOR CONFIGURING A DNA/RNA TARGET PROBE DESIGN”, and incorporated by reference herein in its entirety.

### BACKGROUND

Transcriptomics is the study of transcriptomes, which are the collection of ribonucleic acid (RNA) transcripts present in an organism, group of cells, or individual cell. By identifying the number and distribution of individual transcripts within a cell, transcriptomics can provide researchers with an understanding of which processes are active and which are dormant in the cell. Transcriptomics is often used in genetic counseling, medicine, and to identify species.

One example of a technique used in transcriptomics is fluorescence hybridization. Hybridization experiments use deoxyribonucleic acid (DNA)/RNA probes to peer into the cells of an organ or tissue. A probe refers to a single strand of DNA or RNA that is complimentary to a nucleotide sequence of interest. For example, a probe may take the form of an oligonucleotide (“oligo”), with multiple such probes arranged into a grid in a microarray. The probes bind to the sequence of interest when it is present in the sample and then are caused to fluoresce, thereby allowing researchers to identify the presence and location of the sequence of interest in the sample.

Older fluorescence in-situ hybridization (FISH) techniques involved applying probes that would target only one RNA species at a time. In order to detect multiple target RNA strands within the cell, and to distinguish between cellular background and stray probes, multiple probes may be applied to a sample. Moreover, many different probes had to be applied to a sample in order to identify different RNA species present. An example of this technique is single molecule fluorescence in situ hybridization (smFISH). Although effective, this tended to be a very slow process as each experimental run targeted only a single RNA species out of the hundreds or thousands that might be present in a transcriptome.

More recently, multiplexed FISH techniques have been developed. In these techniques, different probes may be applied simultaneously to the sample, where the different probes each fluoresce in different colors. By reading the colors of the fluorescence, one could study multiple different target RNA sequences at the same time and infer more details about their spatial distribution within the transcriptome. Even so, there are only a limited number of colors that can be distinguished, and so even the best smFISH techniques that applied multiplexing in this manner were able to simultaneously measure about 10-30 RNA species.

In 2015, a different approach FISH transcriptomics, referred to as Multiplexed, Error-Robust FISH (MERFISH), was developed. This combinatorial approach associates a unique barcode with each RNA species, and then reads these barcodes through a series of sequential hybridizations and measurements. More specifically, each RNA species' barcode may be represented as a series of bits (“2”s or “0”s). A probe is applied to a sample and caused to fluoresce. If a given location lights up, it is assigned a “1”; if not, it is assigned a “0”. Then, another probe is applied, and a second bit is read for each location. The number of rounds of imaging to be applied depends on the length of the barcode (and, by association, the total number of RNA species that are being considered). For example, a 16-bit barcode can generate 2¹⁶, or about 65,000, barcodes. This is enough to identify nearly all of the expressed genes in a human cell. The set of binary barcodes and their mappings to specific RNA sequences is referred to as a “codebook.”

A MERFISH probe generally includes three regions. The first region is a targeting region about 30 nucleotides in size that is designed to bind to a portion of an RNA sequence to which it is complimentary. The target regions should include oligonucleotides that bind to their target RNA with high binding efficiency and specificity. It has been found that the target regions work best when designed to cover a relatively narrow range of guanine-cytosine (GC) content and melting temperatures with their target and to have limited homology to other RNAs in the transcriptome (thus reducing the chance that the target region will bind to the wrong RNA). Still further, FISH experiments typically bind a single RNA to multiple probes (rather than binding a single RNA to a single probe), where each probe targets a different portion of the RNA. This increases the brightness of individual RNA spots when performing imaging of the sample to read out the results.

The second region is referred to as a readout region, which helps to speed up transcriptomics experiments. The oligos of this region should have similar melting temperatures and GC content across probes. They also need to be screened for homology to RNAs in the transcriptome of interest. Furthermore, these sequences should have limited homology to each other, so that a readout probe does not bind to the wrong readout sequence.

The third region is a set of priming regions that include DNA primers. DNA primers are short nucleic acid sequences that provide starting points for DNA synthesis. Ideally, the primers in the priming region should have similar melting temperatures, no contiguous stretches of the same nuclueotide longer than three, relatively narrow GC content, and limited homology to each other and to non-priming regions of the probes.

As should be clear from the above description, designing the probes to yield the desired fluorescence combinations for such a large number of possible targets is extremely challenging. The construction of a suitable library of oligos and primers is a complex process that can take a great deal of time (e.g., several days) and significant computing resources (e.g., hundreds of gigabytes of memory usage).

### BRIEF SUMMARY

According to a first embodiment, a probe designer may receive, as an input, a list of genes of interest for a fluorescence in-situ hybridization experiment. The genes of interest may be associated with a transcriptome. Based on the genes of interest, a library of oligonucleotides may be constructed. The oligonucleotides may be configured to bind to at least some of the genes of interest.

Library construction may involve computing possible target regions of the transcriptome, accessing a set of probe creation parameters and assigning values to the probe creation parameters, selecting possible oligonucleotides for the library based on the possible target regions and the values for the probe creation parameters, and adjusting the values for the probe creation parameters. The probe designer may automatically iterate over the selecting and adjusting activities until a stopping condition is met. The thus-constructed library may be stored in a non-transitory computer-readable storage medium.

Because the library construction iterates automatically over the selecting and adjusting activities, probes can be constructed quickly and without the need for expert intervention. Because the selecting and adjusting activities iterate automatically, these operations can be scheduled by a job scheduler. This allows multiple users to work on different probe design projects at the same time and allowing the iterations to be started and stopped. Especially when combined with the front end and back end implementations described in connection with the seventh embodiment below, these automatic iterations can be started at one location and then moved to another.

According to a second embodiment, the automatically iterating may be performed without (and may thus exclude) re-computing the possible target regions of the transcriptome. The present inventors have discovered that the action of re-computing the target regions with each iteration does not improve results by a large margin but does dramatically increase the time and resources required to construct the library. In conventional script-based solutions, it was generally not possible to exclude the step of computing the target regions from subsequent iterations, because the computation script as a whole was run for each iteration. Because the script could not save its state between iterations, the target regions had to be computed afresh on each manual run of the script. By moving to a non-script based approach, the computed target regions could be stored in memory for a subsequent iteration, removing the need to re-compute the target regions at each iteration. Because the automatic iterations exclude computing the possible target regions for the transcriptome, the amount of time, processing resources, and memory requirements for constructing the library are significantly reduced.

According to a third embodiment, the probe creation parameters may include one or more of a number of probes, a probe length, or a probe specificity. By iteratively making changes to these parameters when designing a library, the probe designer can construct a library that is most likely to match genes of interest in the transcriptome.

According to a fourth embodiment, the probe designer may receive the input list of genes of interest from an input parser. The input parser may generate the list of genes of interest by receiving a gene name for one of the genes of interest, access a database that maps common gene names to transcript identifiers, look up the gene name in the database to find a matching transcript identifier for the gene name, and provide the transcript identifier as part of the list of genes of interest. In contrast to conventional script-based solutions that required that a user provide a list of formal transcript IDs as input to the script, searching a database of common gene names makes it easier for users to designate those genes that they are interested in researching. It also reduces the chance of errors in the probe design process by removing ambiguity when designating inputs, thereby leading to probes that more accurately target the intended genes of interest.

According to a fifth embodiment, the input parser may recognize that the received gene name gene maps to multiple possible transcript identifiers, and may offer the multiple possible transcript identifiers as gene synonyms for selection. This solution improves input efficiency by recognizing that the same gene of interest may be referred to by different common names and allowing the user to identify the appropriate transcript ID without the need to manually search highly technical transcript lists.

According to a sixth embodiment, each of the transcript identifiers in the database may be associated with version information. The version information may be omitted from the list of genes of interest provided as input to the probe designer. By avoiding carrying this version information downstream, the memory footprint of the probe designer is reduced without negatively impacting the quality of the library.

According to a seventh embodiment data accessed by the probe designer may be managed by a back end server and computations performed by the probe designer may be performed on a front end server distinct from the back end server. This allows computations to be moved from one front end server to another while relying on the same databases. Furthermore, library creation can be started on one front end server, paused, and then restarted on the same front end server or moved to another one. This separation also allows data and project management to be located on the back end servers, which may be accessed by administrative users. Non-administrative users may be given access to the front end systems to perform library building tasks.

According to an eighth embodiment, the construction of the library may be performed by instructions that are written in a non-script-based language. In contrast to script-based solutions, a non-script based implementation allows for improved capabilities in terms of data management and the ability to save the state of the library construction process. This allows the process to be paused and restarted. Non-script based solutions also allow for automatic iterations, as discussed above, and reduce the need for expert computer programmers in the library construction process. This reduces the cost and complexity of constructing a transcriptomic library.

Any of the above embodiments may be implemented as instructions stored on a non-transitory computer-readable storage medium and/or embodied as an apparatus with a memory and a processor configured to perform the actions described above. It is contemplated that these embodiments may be deployed individually to achieve improvements in resource requirements and library construction time. Alternatively, any of the embodiments may be used in combination with each other in order to achieve synergistic effects, some of which are noted above and elsewhere herein.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. lA is a block diagram depicting exemplary logical modules configured to perform exemplary embodiments.

FIG. 1B is a flow diagram depicting exemplary data exchange in accordance with one embodiment.

FIG. 2 illustrates an exemplary computing architecture suitable for use with exemplary embodiments.

FIG. 3 illustrates an exemplary interface configured to receive a selection of an input list of genes of interest in accordance with one embodiment.

FIG. 4 illustrates an exemplary interface configured to display a list of genes of interest in accordance with one embodiment.

FIG. 5A illustrates an exemplary interface configured to perform automatic iterations to design a library in accordance with one embodiment.

FIG. 5B illustrates an exemplary interface configured to perform automatic iterations to design a library in accordance with one embodiment.

FIG. 5C illustrates an exemplary interface configured to perform automatic iterations to design a library in accordance with one embodiment.

FIG. 6 illustrates an exemplary interface configured to display a report of a generated library in accordance with one embodiment.

FIG. 7 illustrates an exemplary interface for downloading a generated library in accordance with one embodiment.

FIG. 8 is a flow chart depicting logic suitable for practicing exemplary embodiments.

FIG. 9 depicts an illustrative computer system architecture that may be used to practice exemplary embodiments described herein.

### DETAILED DESCRIPTION

Exemplary embodiments described herein provide techniques for efficiently generating a library of oligos and primer sequences for designing a probe in a transcriptomics experiment.

Although conventional solutions for generating such libraries exist, they suffer from a number of problems. Existing solutions tend to be written in scripting languages, which are programming languages configured to be executed in particular types of run-time environments that automate the execution of tasks that would otherwise be manually executed one-by-one. Scripting languages tend to be interpreted (from uncompiled instructions), requiring an interpreter to execute a script directly by translating each statement in the script into a sequence of subroutines, and then translating the subroutines into another language.

Scripting languages have some advantages (e.g., they tend to be platform-independent and so can be executed on the various different types of computing equipment that might be found in a lab), but they also suffer from drawbacks. Scripts require programming skills in order to run, and consume large amounts of processing resources that exceed the capabilities of many laboratories. Accordingly, building a library can take a great deal of time and requires a programmer with some expertise (thus increasing the costs of running transcriptomics experiments).

The present solution implements a non-script based approach. In addition to requiring fewer resources and being easier to operate by non-programmers, this approach allows for further improvements that can significantly decrease processing complexity, resource requirements, and the time required to generate a library. These solutions can be implemented individually for improvements in these areas, but can also be implemented together to yield increased synergistic effects.

For example, as alluded to above a probe designer requires an input list of genes of interest (“GOT”) in order to design a suitable probe library. Conventional script-based solutions operate on GOIs provided as input; the correct selection of these GOIs is therefore up to the user. However, GOI databases tend to be rigid in their structure and naming conventions. A gene commonly known by one name might be represented by different transcript IDs in different databases (or different common names might represent the same transcript ID). This means that a user must manually search each database and review the resulting GOIs to ensure that they are building probes for the correct genes. This procedure is not generally a part of the probe design script, but must be performed beforehand in order for the probe designer to operate.

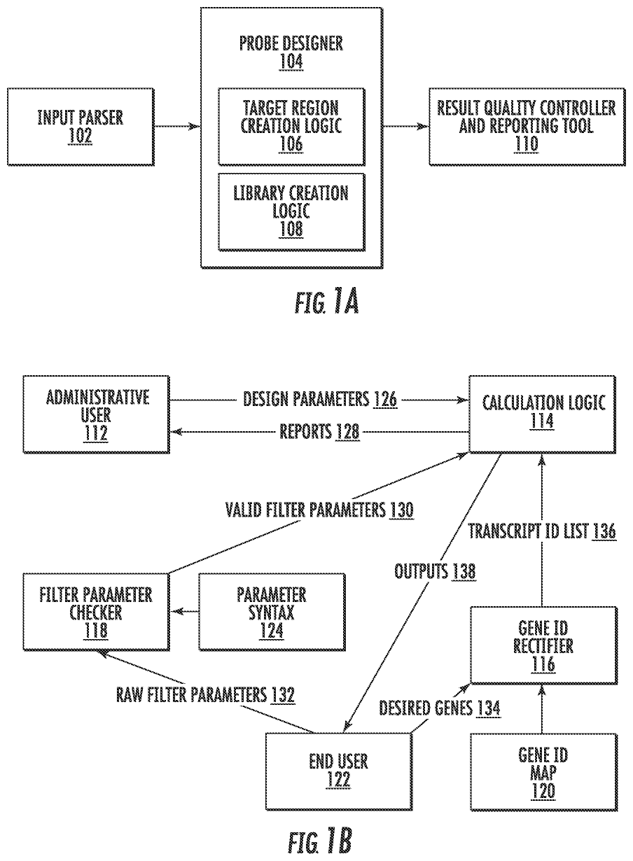

As shown in FIG. 1A, the solution described herein includes an input parser 102 that recognizes various forms of inputs and converts them into a formal GOI names using internally-constructed databases. Thus, it is not required that a user manually find the GOIs in external databases and verify that the correct GOIs are manually input into the script. This improvement greatly simplifies the input process into the probe designer 104.

The internally-constructed databases include common gene names that map to transcript identifiers. These internally-constructed databases may combine multiple transcript sources and may map multiple different common names to a given transcript identifiers. Furthermore, the input parser 102 may recognize different gene synonyms. If a user enters a particular name for a gene (e.g., “gene A”), the input parser 102 may recognize that the named gene may read on different genes or gene subsets (e.g., “gene A-1,” “gene A-2,” etc.), and may automatically search these synonyms.

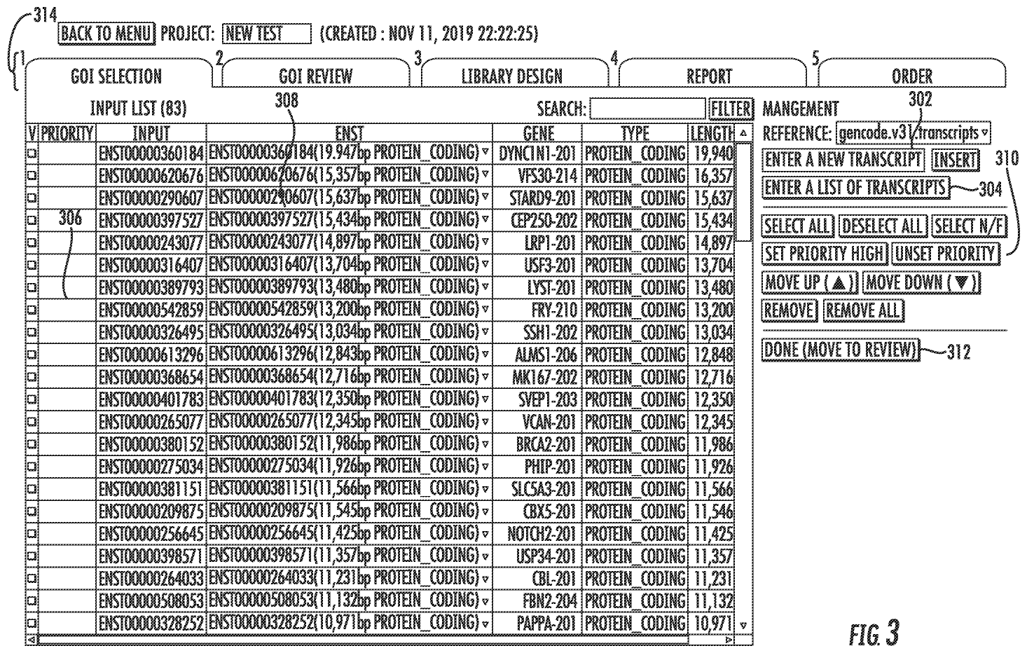

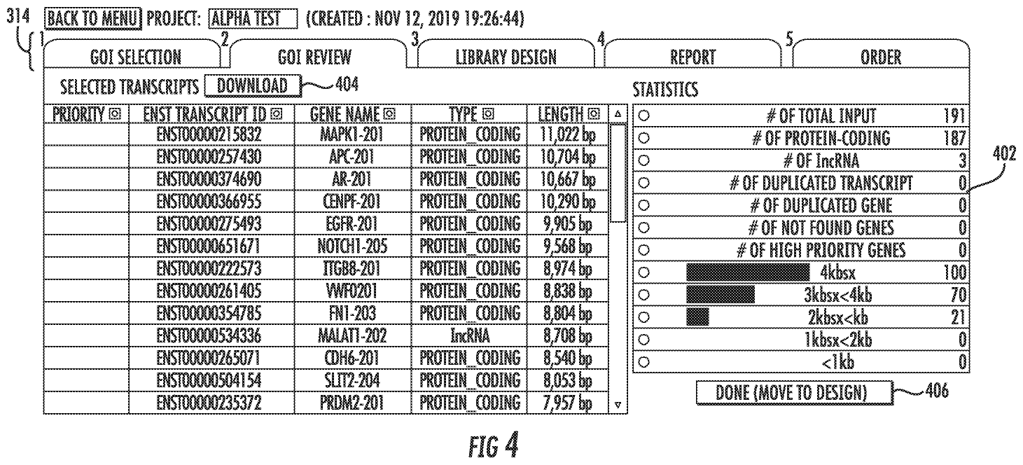

By querying these internally-constructed databases using common gene names and synonyms, the process of setting up the inputs to the probe designer 104 is simplified and made faster and more efficient. Moreover, the accuracy of the probes (in targeting the desired GOIs) is improved because the internally-constructed databases reduce ambiguity when selecting GOIs. An example of an interface for the input parser 102 used to select GOIs is depicted in FIG. 3. Once selected, the transcript IDs can be reviewed in a GOI review interface as illustrated in FIG. 4, thereby allowing for improved quality control.

Script-based systems also typically have restrictions on the maximum amount of data that they can save. By moving to a non-script-based approach, limitations on data saves are reduced or eliminated. Consequently, probe designs can be saved on a project-by-project basis. Users can stop or start a project as desired, saving the results at any point (including completed results) for future processing. It also provides the capability for users to access their results from any location, not just the local computer performing the probe design. This means that the work can be split between front-end and back-end systems so that, e.g., a back-end server can be used for database management while a front-end server performs the necessary calculations to design the probes. Work can then be moved from one front-end system to another so that the process can be started in one location and then moved to another, or so that results can be modified or re-run at new locations.

Furthermore, conventional probe designers may require several iterations in order to obtain an optimal result. Generally, no perfect probe design will exist for a given sample and set of genes of interest; a user's goal may be to design a set of probes that are configured to detect the most genes of interest possible given the constraints of the experiment. When a probe designer outputs a given library or design, it may be desirable to revisit certain design parameters and run the probe design process again in order to capture the greatest number of targets at the highest level of specificity.

Conventional script-based solutions rely on manually iterating over these steps, which can be inefficient and take a great deal of time. Even if automated however, the design of conventional scripts tends to make the probe design process more complicated than it might otherwise be. As shown in FIG. 1A, the exemplary probe designer 104 includes target region creation logic 106 for computing the possible target regions in the transcriptome and library creation logic 108 for selecting possible genes/oligos for inclusion in the library. Conventionally, target region creation and library creation both involved several steps that were part of the iterations and thus had to be run over and over again. However, the present inventors have identified that certain parts of the target region creation logic 106 do not necessarily need to be performed with each iteration, and have accordingly moved the portions that do require iteration into the library creation logic 108. As a result, the probe designer 104 can iterate over the library creation logic 108 without iterating over the target region creation logic 106, resulting in significant savings of time, memory, and processing resources.

A system implementing the exemplary embodiments described above have been tested against a conventional script-based solution, and the results were compiled for purposes of illustration and comparison. In one test, the conventional script-based solution required 146 GB of memory usage and required approximately two days to run (this excludes the process of input GOI selection, which as noted above was a separate process and could add an additional 12 hours to the required processing time). Using the embodiments described herein, memory requirements dropped to 18 GB and the runtime dropped to 4-6 minutes (again, excluding the input process of transcript ID selection, which was itself reduced from about 12 hours to 3-4 hours).

Furthermore, conventional tools provide output results, but do not offer much in the way of quality control capabilities. In exemplary embodiments, a result quality controller and reporting tool 110 generates an output report that provides a user with an at-a-glance overview of a constructed library to see if it satisfies their expectations. A visual diagram may show the construction of the probes for ease of use.

In addition, the result quality controller and reporting tool 110 may perform post-library quality control by performing a random blast check of constructed probes and encoding the probe specificity. This allows the user to ensure that the constructed library did not alter the probe specificity iteration-to-iteration.

FIG. 1B is a flow diagram depicting exemplary data exchange in accordance with one embodiment.

An administrative user 112 may provide design parameters 126 to calculation logic 114. The calculation logic 114 may be responsible for determining, based on an input transcript ID list 136, a probe design library including (e.g.) oligos and primers configured to bind to the genes of interest represented by the transcript ID list 136. The design parameters 126 may include configuration options for configuring the calculation logic 114, such as the size and layout of the codebook used during the library construction process, as well as the number of blank entries in the codebook. The administrative user 112 may also receive reports 128 about the performance of the calculation logic 114 and/or the databases that the calculation logic 114 interacts with, thus allowing the administrative user 112 to adjust the design parameters 126 to improve performance.

The calculation logic 114 may operate on a list of genes of interest. To that end, the calculation logic 114 may receive, as an input, a transcript ID list 136, which includes a list of the genes of interest that the calculation logic should be applied to. The transcript ID list 136 may include identifiers for the genes of interest in a format recognizable by the calculation logic 114; this may include transcript IDs formatted and designated according to an accepted standard, such as a scientific standard.

This may, however, be somewhat restrictive for the end user 122, who may not know the transcript IDs for all possible genes of interest, or who may refer to certain genes of interest by their common names. Accordingly, a gene ID rectifier 116 may be provided, which translates common gene names into transcript IDs. In order to achieve this effect, the gene ID rectifier 116 may accept an input list of desired genes 134 from the end user 122 and may match the desired genes 134 to the transcript IDs using a gene ID map 120. The gene ID map 120 may include one or more common names for each desired gene, and may recognize gene synonyms. If there is any ambiguity as to which transcript ID should be used with a particular desired gene, the gene ID rectifier 116 may present a prompt to the end user 122 to allow the end user to resolve the ambiguity.

The desired genes 134 may be provided to the gene ID rectifier 116 through a wizard or user interface (e.g., as shown in FIG. 3). Alternatively or in addition, the desired genes 134 may be provided via an external file or database. Once each desired gene has been mapped to a transcript identifier, the gene ID rectifier 116 may output the transcript ID list 136 to the calculation logic 114.

The goal of the calculation logic 114 is to generate an oligo library and list of primers as outputs 138 to be provided to a computing device of the end user 122. However, given the constraints identified in the background section above, this may be a very complicated task. It may be important to filter and adjust the library in order to achieve desired results based on parameters that place limits on the design of the probe (such as the acceptable melting temperature range, the length of the possible probes, suitable GC content, maximum melting temperature range for off-target sequences, the maximum acceptable run length of the same nuecleotide, etc.).

Accordingly, the user may provide filter parameters to the calculation logic 114. In order to allow for more efficient and accurate entry of the filter parameters, a set of raw filter parameters 132 may be provided to a filter parameter checker 118, which accepts the raw filter parameters 132 and checks their syntax and structure. For example, the filter parameter checker 118 may consult a database of parameter syntax 124. The filter parameter checker 118 may ensure that the parameters are formatted correctly and are eligible to be applied together, and may then output valid filter parameters 130 to the calculation logic 114.

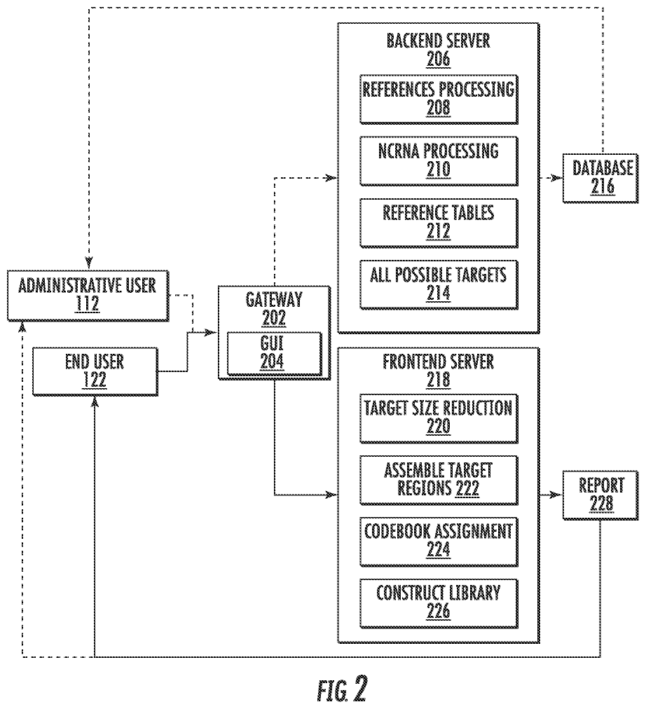

Various tasks described above may be performed at different locations in a computing architecture. FIG. 2 depicts an example of such an architecture suitable for use with exemplary embodiments.

The administrative user 112 and the end user 122 may each interact with the architecture by accessing a gateway 202 that presents a GUI 204. Examples of suitable GUIs 204 are presented in FIGS. 3-7.

The architecture includes a frontend server 218 and a backend server 206, each accessible to the user base in different ways depending on their roles. For instance, the backend server 206 is generally responsible for maintaining a database 216 that includes various data items used to build an oligo library, and is generally accessible primarily to the administrative user 112. The frontend server 218 may be responsible for performing calculations according to the calculation logic 114, and may iterate over the actions shown in FIG. 2 (e.g., assembling the target regions 222, codebook assignment 224, and library construction 226 actions detailed below). The frontend server 218 may be accessed by both the administrative user 112 and the end user 122. The frontend server 218 may make use of the information in the database 216 maintained by the backend server 206. The frontend server 218 may generate one or more reports, which may include library output reports and/or performance reports. The reports may be provided to the administrative user 112 and/or the end user 122, as appropriate.

The backend server 206 may accept the input transcript ID list 136, and may perform references processing 208 to identify reference genes among the genes of interest. During references processing 208, all the sequences of the transcriptome (regardless of their transcript ID) may be bioinformatically fragmented into subsequences and assigned a value (e.g., a hash) based on their composition. In other words, each fragment with a unique sequence composition will have a corresponding unique hash assigned to it. The hashes may be rearranged into a data table with a corresponding number of occurrences within transcriptome. This may allow the probes to be targeted based on the frequency at which the various sequences occur in the transcriptome.

The backend server 206 may also identify non-coding RNA (ncRNA) among the genes of interest via ncRNA processing 210. ncRNA processing follows the same general procedure as references processing 208 but uses ncRNA (non-coding RNA) sequences. Reference processing and ncRNA processing may be performed together in a single run-through of the transcriptome sequences.

Based on the information obtained in 208, 210, the backend server 206 may compute all possible target regions 214 in a transcriptome that are available for study using a set of reference tables 212.

The output of the backend server 206 may be provided to the database 216. The database 216 may store the transcript ID list 136, the results of the references processing 208 and ncRNA processing 210, the references reference tables 212, and/or the possible target regions. The database 216 may be generated according to design or input parameters specified by a user. The parameters may include target length (bp), melting temperature, G/C contents, sequence specificity, and others. The algorithm may recalculate the parameter value for each fragment using values in a data table. In other words, the database may contain corresponding parameter information of all fragments that are recalculated according to a given target length using the data table from references processing 208.

The frontend server 218 may use the information in the database 216 to compute an oligo library.

For example, the frontend server 218 may retrieve the possible target regions from the database and perform target size reduction 220, for example by factoring out ncRNA from the target regions and filtering the target regions based on fragments per kilo base per million mapped reads (FPKM), or another suitable normalized measurement of transcript abundance. Once the target size has been reduced, the frontend server 218 may assemble target regions 222 of the probes for targeting by the available oligos.

FIG. 3 illustrates an exemplary interface configured to receive a selection of an input list of genes of interest in accordance with one embodiment. The interface allows a user to input a name for a gene of interest and covert it to a transcript identifier suitable for input into the calculation logic 114.

The interface includes an entry field 302 into which a user can enter the common name or some other designator indicating a gene of interest, such as a Gene Symbol, ENSG, an Ensembl gene ID, an Ensembl transcript ID, etc. Wildcard functionality may be employed to search the mappings (e.g., entering “BR*” into the entry field 302 may search the mappings for every gene name that starts with the letters “BR”). Alternatively or in addition, the user may select the import button 304 to import a list of transcripts (e.g., in spreadsheet, comma separated value, or some other suitable form).

The system may search a database of mappings that converts the common name or other identifier into a suitable transcript ID (e.g., an Ensembl transcript ID), if necessary. The available genes of interest may be shown in an input list 306, and any transcript IDs that match the search may be highlighted in the input list 306. Within the input list 306, a user can select one of the transcript identifiers that matches their intended gene of interest using a transcript selector 308.

The user may be more interested in targeting certain genes of interest in a sample than others. Accordingly, once a transcript ID is selected using the transcript selector 308, the user can adjust the priority assigned to the transcript ID using priority adjustment elements 310. When designing the probes, the system may account for the priority to attempt to target higher priority genes over lower priority genes.

After a user has selected all desired genes of interest, the user can advance to the next interface by selecting a done element 312. Alternatively or in addition, the user may navigate between the various interfaces depicted using navigation tabs 314.

FIG. 4 illustrates an exemplary interface configured to display a list of genes of interest in accordance with one embodiment. This interface allows the user to review the genes of interest selected using the interface depicted in FIG. 3. In this display, a statistics column 402 displays relevant information about the selected genes, such as the total number of genes, the number of coding and non-coding genes among the selections, the number of genes that could not be found in the search, the number of genes that were duplicated in the search, the kilobase measurements of the genes in the list, etc.

Based on the information in the statistics column 402, the user may wish to return to the selection interface of FIG. 3, which can be done using the navigation tabs 314. Alternatively, if the user is satisfied with their selections, they can export the converted list using the download element 404. The user can also use the done element 406 to move to the next interface to design probes targeted to the selected genes of interest.

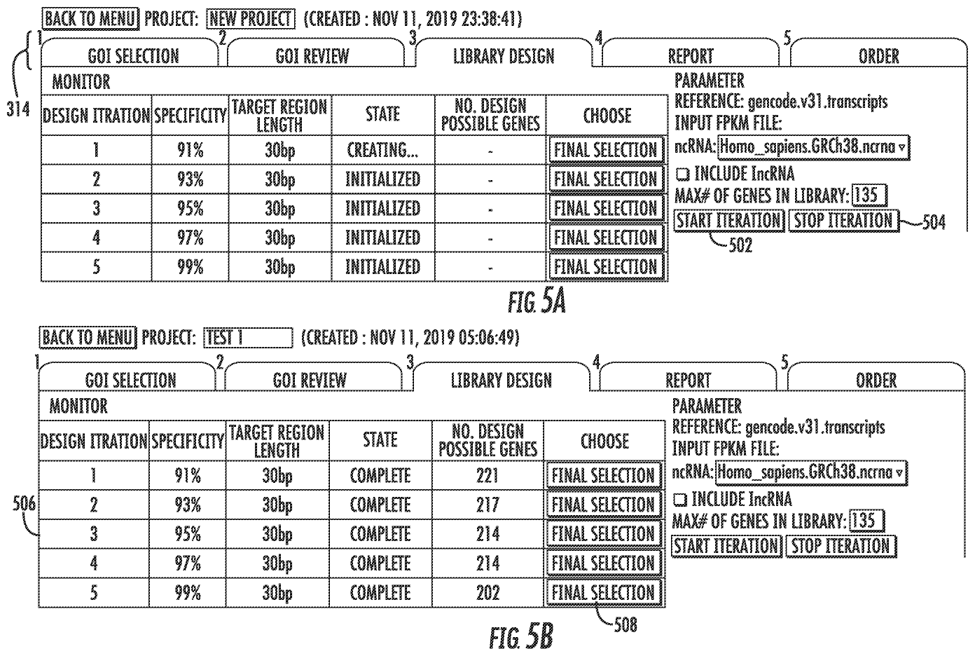

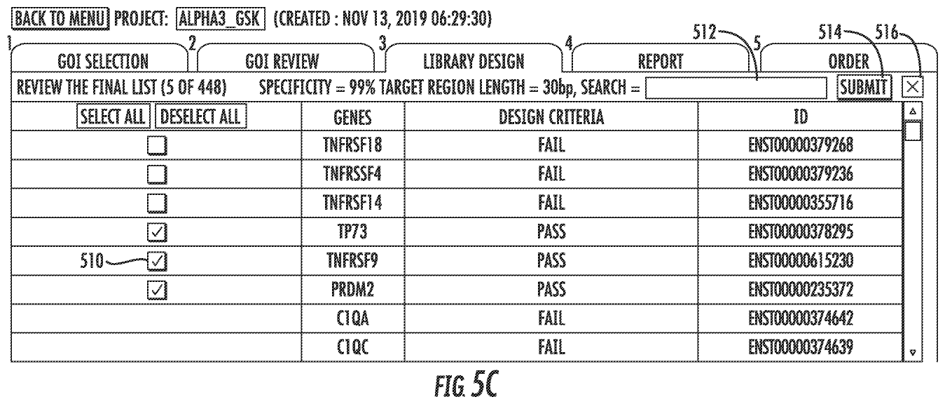

FIG. 5A illustrates an exemplary interface configured to perform automatic iterations to design a library in accordance with one embodiment. The iteration process involves selecting an initial set of design parameters for the probes, such as the number of probes to be used (options may include, e.g., 78, 60, and 50 probes), the length of the probes, and the specificity of the probes. The design parameters may be predefined and/or may be adjusted by an administrative user 112 by transmitting new design parameters 126 to the calculation logic 114.

A user can start the automatic iteration process by selecting the iteration start element 502. Upon selecting the iteration start element 502, the system reads the initial design parameters 126 and attempts to optimize an oligo and primer library for the probes in order to (e.g.) maximize the number of design possible genes. “Design possible genes” refers to the genes from the transcriptome that can be targeted by (e.g., will bind to) the DNA/RNA sequence in the set of probes in the experiment. The system may define a maximum number of design possible genes that can be targeted (e.g., a number, such as 136, corresponding to the maximum possible size of the codebook), although a user might specify that a number fewer than the maximum should be targeted (e.g., 100). This might allow the user to increase the specificity for the set of probes (targeting fewer genes, but with more specificity). An example of logic for building a probe in one of the iterations is the MERFISH software developed at Harvard University of Boston, Massachusetts.

When an iteration finishes, the system may select a new set of design parameters and attempt to optimize a new oligo and primer library for the probes. The process may continue until a predetermined stopping condition is met (such as reaching a maximum number of desired iterations, exceeding a predefined threshold calculation time, exhausting the possible design parameters, etc.). In one test of an exemplary embodiment, each iteration took approximately 4 minutes. A user can also end an iteration early by selecting the iteration stop element 504.

The current statuses of the scheduled iterations are shown as iteration summaries 506. The iteration summaries 506 may summarize the design parameters for the probes (e.g., the specificity and target region length of the probes), as well as whether an iteration is in progress, scheduled but not yet started, paused, completed, etc. A user can also select an iteration and cause it to execute out-of-order, and/or drag iterations to rearrange the order in which they will be executed. When an iteration is complete, the number of design possible genes for the oligo/primer library as calculated in that iteration may also be shown in the iteration summaries 506.

For instance, FIG. 5B depicts the iteration interface after all scheduled iterations have completed. As each iteration finishes, the number of design possible genes are displayed, and a selection element 508 appears next to each iteration to allow the user to select a specific iteration to select the final genes for the library.

This selection step is particularly important if the number of design possible genes from iteration is greater than the maximum number of genes allowed in the codebook. If the codebook cannot accommodate all of the design possible genes, then the user may wish to use the navigation tabs 314 to change the problem setup (and/or change the design parameters) until all of the design possible genes can be accommodated. If the genes of interest are changed on the GOI selection interface, it may be necessary to repeat any iterations that have already completed.

After selecting the iteration to be used to build the library, a user may be presented with a review interface showing the results of the iteration including the design possible genes, as shown in FIG. 5C. A user can use the gene library selector 510 to include or exclude design possible genes from the final oligo/primer library. If a user is interested in a particular gene, the user can search for the gene in the gene search field 512. When the user is satisfied with the primer/oligo library, the user can accept the results by selecting the submit element 514, which then generates and displays a reporting interface.

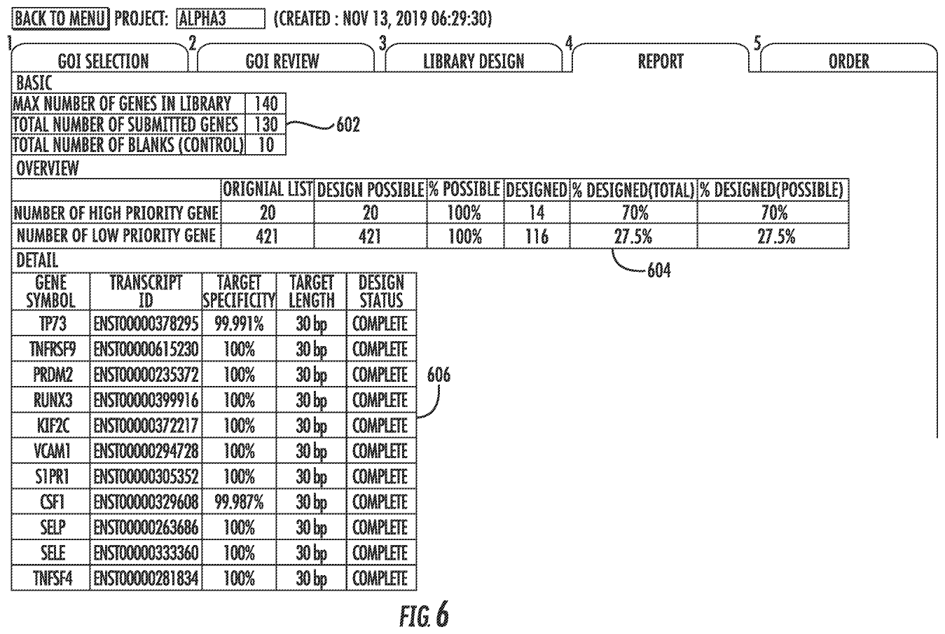

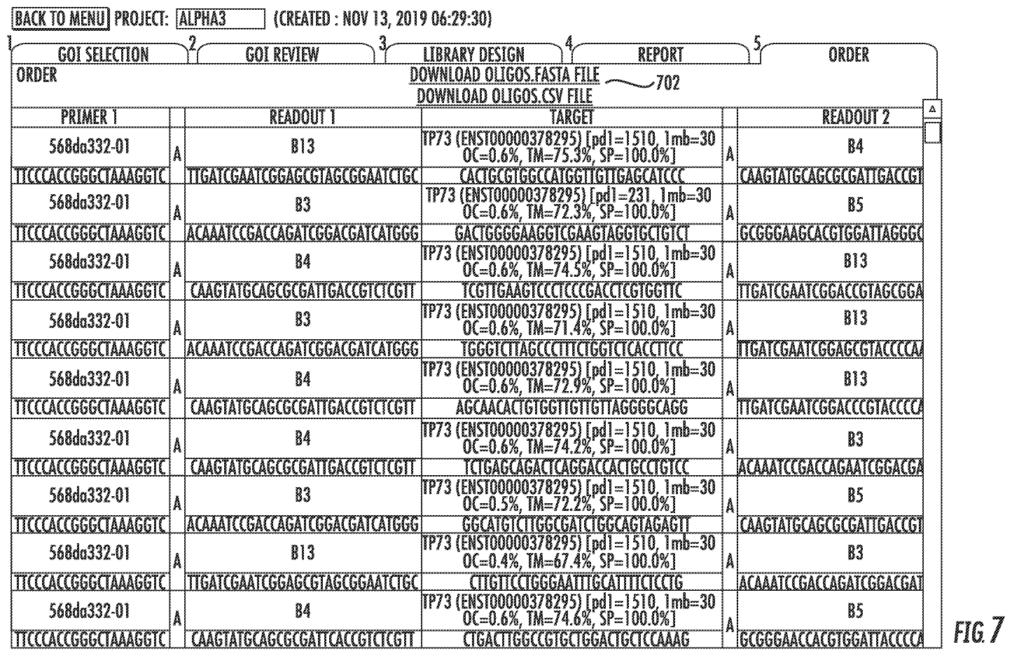

An example of such a reporting interface is depicted in FIG. 6. The reporting interface summarizes information about the library so that a user can decide whether to download the library, or return to an earlier step and redesign the library.

For instance, the reporting interface may provide a basic information summary 602 showing the maximum number of genes that can be included in the library (as defined, e.g., by the size of the codebook), the total number of submitted genes that the user selected in the interface of FIG. 5C, and the total number of blanks that serve as controls for the probes.

The reporting interface may also show a priority breakdown 604, providing details about genes that were prioritized or de-prioritized using the priority adjustment elements 310. For example, FIG. 6 shows a priority breakdown 604 that summarizes high priority and low priority genes (although more granular breakdowns are also possible0. The priority breakdown 604 describes, for each priority category, the number of such genes of interest in the original list, the number of design-possible genes reported by the selected iteration, the ratio or percentage of design possible genes to originally requested genes, the number of designed genes (e.g., genes selected for inclusion in the library in FIG. 5C), the percentage or ratio of designed genes to the total number of originally-selected genes of interest, and the ratio or percentage of designed genes to design possible genes.

Furthermore, a gene details 606 table may provide information about the design possible genes for the selected library, as well as the gene's status in the library (e.g., whether it was selected for inclusion in the interface shown in FIG. 5C).

If the user is not satisfied with the results reported in the interface, the user can revert to a previous step to change the library design and repeat the iterations. Otherwise, the user may advance to an order interface, an example of which is shown in FIG. 7.

The order interface optionally displays the complete library result in a table, and also provides a download element 702 allowing the user to save the library to an appropriate data structure On a computer-readable medium. The structure may store information about only the designed genes, or might store information about both the design possible genes and the designed genes. In some embodiments, the library may include the designed genes, whereas the combination of designed genes and design possible genes may be stored in a separate repository. The structure may then be used in order to assemble probes according to the library.

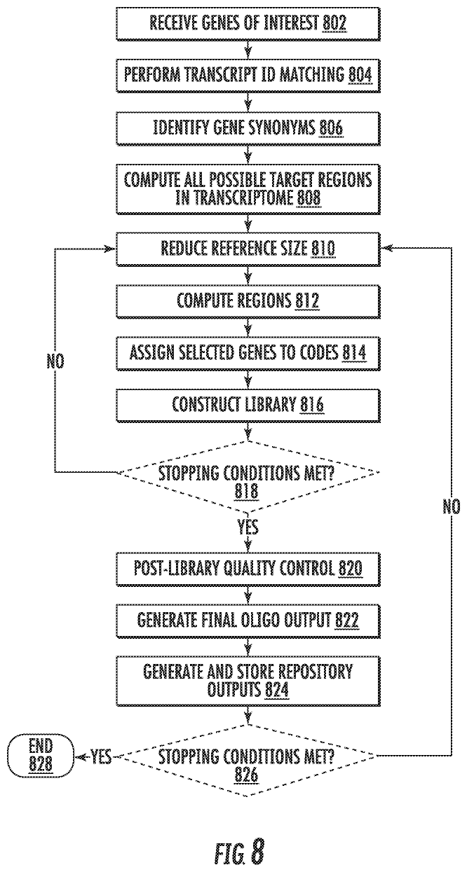

The above-described interfaces and processes may be implemented by suitable logic, as illustrated for example in FIG. 8. The logic may be stored as instructions on a non-transitory computer-readable storage medium and may be configured to cause a processing circuit to perform the described operations. The storage medium and hardware may be co-located, or the instructions may be stored remotely from the processor. Collectively, the medium and processor may form a system.

At block 802, the system may receive a list of genes of interest. The genes of interest may be genes for a FISH probe. The list of genes of interest may be retrieved from a storage location (such as a computer file), may be entered in an interface or wizard, or a combination of the two (among other possibilities).

The genes of interest may be associated with a transcriptome, which may also be specified. The genes of interest may be part of the transcriptome, and may be targeted by the FISH probe during a transcriptomics experiment.

At block 804, the system may perform transcript ID matching. In this block, the system compares each of the genes on the list of genes of interest from block 802 to a database. The database may map common gene names or other RNA designators to formal transcript identifiers. The system may query the database for each of the genes of interest and receive, in response, the formal transcript identifiers. If there is any ambiguity as to which transcript identifier is intended by a given input, the system may present a list of options (e.g., a drop-down list as shown in FIG. 3) and allow the user to select the correct transcript identifier. Based on the user selections and the output from the database, the system may generate a list of transcript identifiers.

At block 806, which may occur in parallel with block 804, the system may identify any gene synonyms. As used herein a “gene synonym” refers to one of multiple names that a given gene is commonly known by. For example, gene A may commonly be known by names A-1, A-2, etc. A user may inadvertently enter gene synonyms as genes of interest, thinking that the synonyms refer to different genes. This can be undesirable, since the user may think that they are incorporating more genes of interest into the probe design than they actually are; if they were aware that the synonyms referred to the same gene, they might include more, different genes of interest.

The system may identify the gene synonyms by querying a synonym database, list, or other suitable structure that maps gene synonyms into a single transcript identifier. If two or more user-entered genes of interest map to the same transcript identifier, the system may flag the genes of interest as synonyms and offer an option for the user to select the common transcript identifier referred to by the synonyms and proceed with more gene of interest selections.

At block 808, the system may compute all possible target regions in the transcriptome (i.e., the transcriptome to which the FISH probe is to be targeted, as specified for example in block 802). The “target regions” include the transcripts in the transcriptome to which probes may be targeted. In block 808, the system may consult a database or other data structure that includes the transcripts in each given transcriptome. The system may query the database based on the transcriptome, and may receive in response a list of transcripts representing the possible target regions in the transcriptome.

Each probe that is being designed may be targeted to a particular target region. Some probes may read on more than one target region, and in some cases multiple different probes might read on a single target region. When designing a probe (or set of probes) for a transcriptomics experiment, it is important to take into account factors such as the number of probes being applied in the experiment, the length of each probe, or the specificity of each probe, the binding efficiency of each probe, the guanine-cytosine (GC) content of each probe, and the melting temperature of each probe. These factors may be considered input parameters, and may be provided by a user via an interface or may be set to a default value. In some embodiments, a range of values may be specified for the input parameters, or a set of values, or an acceptable amount of variance. In some cases, default values may be applied. For example, the system might specify a set of values {78, 70, 60, 50} that can be used for the number of probes; this set of values will be iterated over at block 818. The system might also adjust the probe length, acceptable melting point, and/or specificity within an acceptable range at each iteration.

The next goal is to define the probes in a set of probes for the experiment by selecting suitable DNA/RNA sequences that can bind to the target regions. To this end, at block 812, the system may compute target regions for the probes based on the above-identified input parameters. Working within the constraints imposed by the input parameters, the system may attempt to maximize the number of design possible genes in the transcriptome (as discussed above in connection with FIG. 5) targeted by the probe set. This can be achieved, for example, by searching an n-dimensional search space for a combination of n probes that meet the requirements of the input parameters and maximizes the number of design possible genes.

Among other factors, the system may consider a desired signal-to-noise ratio (or a proxy for the SNR). For example, it may be possible for a set of 100 probes to target 100 different target regions of a transcriptome, but this may result in a great deal of noise in the experiment (since each probe will only identify one target region, and the intensity of the fluorescence of the probe may not be picked up). At the other extreme, all 100 probes might target a single target region, which would result in a very strong signal but only a small number of targetable target regions. The system may attempt to balance the SNR of the experiment so that it does not fall below a predetermined or user-specified threshold In some embodiments, the user may directly specify the number of probes to assign to some or all target regions via an interface.

The result of block 812 may be a set of selected genes for the probes. At block 814, the system may assign selected genes to codes. In this step, the system builds a codebook that maps the selected genes to available barcodes; the number of available barcodes depends on the size selected for the barcode, in bits. The longer the barcode, the more genes can be stored. However, the downside to longer barcodes is that more probes must be applied and more images recorded in order to characterize all the genes in a given transcriptome. The sizes of the codebook and barcodes are typically predetermined and fixed, so at this block the available barcodes are computed and assigned to the genes. Various techniques for assigning barcodes are known, and one of ordinary skill in the art will appreciate that any of the available codebook-building techniques may be applied at block 814.

At block 816, the system may construct a library based on the activities performed in blocks 810-814. For example, the system may construct a library using the process described above in connection with FIGS. 5A-5C and based on the genes selected at block 812. The result of the library construction process for a given set of input design parameters may be a list of oligos and primers that may be used for a set of probes, along with the number of design possible genes that would result from using the identified oligos and primers on a set of probes. The library construction process may entail calculating the values to be used in the interfaces depicted in FIGS. 5A-7. A suitable process for selecting appropriate oligos and primers is described in connection with the MERFISH software referenced above.

At decision block 818, the system may determine if a set of stopping conditions has been met. For example, the system may iterate over different combinations of specified input design parameters until all combinations have been considered (or a specified subset of the combinations have been considered). The system might also or alternatively perform a predetermined or selected number of iterations, iterate for a specified or predetermined period of time, etc.

If the decision at decision block 818 is “NO” (i.e., more iterations remain to be performed), then the system may revert to block 810 and begin recomputing the library using a different set of design parameters. Notably, the system does not return to block 808 (and therefore does not recompute all possible target regions in the transcriptome at each iteration). Although this step was typically performed in each iteration in the past (because such iterations tended to be manual and could not store information from previous runs to be relied upon later), the present inventors have found it to be unnecessary to the automatic iterations performed by a non-script-based implementation as described herein. Eliminating the calculations of block 808 in subsequent iterations saves a significant amount of time and computing resources.

If the decision at decision block 818 is “YES” (i.e., no more iterations remain to be performed), then processing may proceed to block 820 and the system may perform post-library quality control. This may involve performing a random blast check of the probes and encoding the probe specificity.

At block 822, the system may generate a final oligo/primer output. The final oligo/primer output may include the oligos and primers selected for inclusion in the library at block 816, and may optionally exclude any design possible genes that were not selected for inclusion. The system may store the oligo/primer output as a library in any suitable data structure. For example, the library may be saved as a comma-separated value list, a spreadsheet, a database, a table or matrix, a list, as web code for displaying a web page, or any other suitable format. According to one embodiment, the complete list of oligos and primers, including both design possible genes and designed genes, may be saved in a repository at block 824. The repository may include the final selected oligos that were designed in the library, the sum total of the possible oligos (e.g., the design possible oligos, including those that were not selected for inclusion in the library), the list of all possible primer sequences, the list of the primer sequences selected for use, and the readout sequences to be used on the readout regions of the probes.

At decision block 826, the system may determine if another set of stopping conditions have been met. If so, then processing proceeds to block 828 and terminates; if not, processing may revert to block 810. For example, if the user is unsatisfied upon reviewing the final output library, the user may change some of the genes of interest or design parameters, which might require the system to iterate again over the new selections. If the user is satisfied, the user can download the final oligo/primer output and/or the repository from its storage location.

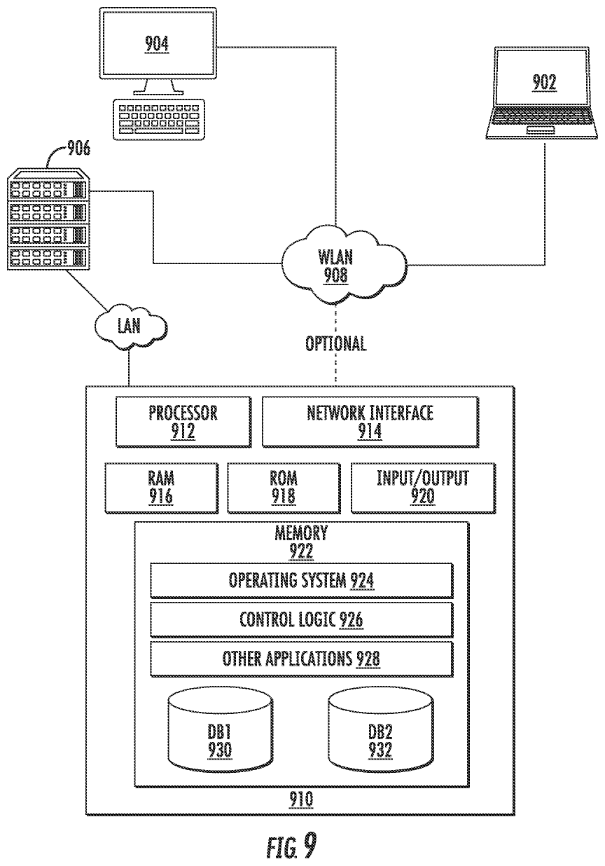

FIG. 9 illustrates one example of a system architecture and data processing device that may be used to implement one or more illustrative aspects described herein in a standalone and/or networked environment. Various network nodes, such as the data server 910, web server 906, computer 904, and laptop 902 may be interconnected via a wide area network 908 (WAN), such as the internet. Other networks may also or alternatively be used, including private intranets, corporate networks, LANs, metropolitan area networks (MANs) wireless networks, personal networks (PANs), and the like. Network 908 is for illustration purposes and may be replaced with fewer or additional computer networks. A local area network (LAN) may have one or more of any known LAN topology and may use one or more of a variety of different protocols, such as ethernet. Devices data server 910, web server 906, computer 904, laptop 902 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data--attributable to a single entity--which resides across all physical networks.

The components may include data server 910, web server 906, and client computer 904, laptop 902. Data server 910 provides overall access, control and administration of databases and control software for performing one or more illustrative aspects described herein. Data serverdata server 910 may be connected to web server 906 through which users interact with and obtain data as requested. Alternatively, data server 910 may act as a web server itself and be directly connected to the internet. Data server 910 may be connected to web server 906 through the network 908 (e.g., the internet), via direct or indirect connection, or via some other network. Users may interact with the data server 910 using remote computer 904, laptop 902, e.g., using a web browser to connect to the data server 910 via one or more externally exposed web sites hosted by web server 906. Client computer 904, laptop 902 may be used in concert with data server 910 to access data stored therein, or may be used for other purposes. For example, from client computer 904, a user may access web server 906 using an internet browser, as is known in the art, or by executing a software application that communicates with web server 906 and/or data server 910 over a computer network (such as the internet).

Servers and applications may be combined on the same physical machines, and retain separate virtual or logical addresses, or may reside on separate physical machines. FIG. 9 illustrates just one example of a network architecture that may be used, and those of skill in the art will appreciate that the specific network architecture and data processing devices used may vary, and are secondary to the functionality that they provide, as further described herein. For example, services provided by web server 906 and data server 910 may be combined on a single server.

Each component data server 910, web server 906, computer 904, laptop 902 may be any type of known computer, server, or data processing device. Data server 910, e.g., may include a processor 912 controlling overall operation of the data server 910. Data server 910 may further include RAM 916, ROM 918, network interface 914, input/output interfaces 920 (e.g., keyboard, mouse, display, printer, etc.), and memory 922. Input/output interfaces 920 may include a variety of interface units and drives for reading, writing, displaying, and/or printing data or files. Memory 922 may further store operating system software 924 for controlling overall operation of the data server 910, control logic 926 for instructing data server 910 to perform aspects described herein, and other application software 928 providing secondary, support, and/or other functionality which may or may not be used in conjunction with aspects described herein. The control logic may also be referred to herein as the data server software control logic 926. Functionality of the data server software may refer to operations or decisions made automatically based on rules coded into the control logic, made manually by a user providing input into the system, and/or a combination of automatic processing based on user input (e.g., queries, data updates, etc.).

Memory 1122 may also store data used in performance of one or more aspects described herein, including a first database 932 and a second database 930. In some embodiments, the first database may include the second database (e.g., as a separate table, report, etc.). That is, the information can be stored in a single database, or separated into different logical, virtual, or physical databases, depending on system design. Web server 906, computer 904, laptop 902 may have similar or different architecture as described with respect to data server 910. Those of skill in the art will appreciate that the functionality of data server 910 (or web server 906, computer 904, laptop 902) as described herein may be spread across multiple data processing devices, for example, to distribute processing load across multiple computers, to segregate transactions based on geographic location, user access level, quality of service (QoS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). various aspects described herein may be embodied as a method, a data processing system, or a computer program product. Therefore, various functionalities may be embodied in whole or in part in software, firmware and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A computer-implemented method comprising:
receiving, as an input at a probe designer, a list of genes of interest for a fluorescence in-situ hybridization experiment, the genes of interest associated with a transcriptome;
constructing a library of oligonucleotides configured to bind to at least some of the genes of interest, the constructing comprising:
computing possible target regions of the transcriptome,
accessing a set of probe creation parameters and assigning values to the probe creation parameters;
selecting possible oligonucleotides for the library based on the possible target regions and the values for the probe creation parameters,
adjusting the values for the probe creation parameters, and
automatically iterating over the selecting and adjusting until a stopping condition is met; and
storing the constructed library in a non-transitory computer-readable storage medium.

2. The method of claim 1, wherein the automatically iterating excludes re-computing the possible target regions of the transcriptome.

3. The method of claim 1, wherein the probe creation parameters include one or more of a number of probes, a probe length, or a probe specificity.

4. The method of claim 1, further comprising generating the list of genes of interest by:
receiving a gene name for one of the genes of interest;
accessing a database that maps common gene names to transcript identifiers;
looking up the gene name in the database to find a matching transcript identifier for the gene name; and
providing the transcript identifier as part of the list of genes of interest.

5. The method of claim 4, further comprising recognizing that the received gene name gene maps to multiple possible transcript identifiers, and offering the multiple possible transcript identifiers as gene synonyms for selection.

6. The method of claim 4, wherein each of the transcript identifiers in the database is associated with version information, and the version information is omitted from the list of genes of interest provided as input to the probe designer.

7. The method of claim 1, wherein data accessed by the probe designer is managed by a back end server and computations performed by the probe designer are performed on a front end server distinct from the back end server.

8. The method of claim 1, constructing the library is performed by instructions that are written in a non-script-based language.

9. A non-transitory computer-readable storage medium, the computer-readable storage medium including instructions that when executed by a computer, cause the computer to:
receive, as an input at a probe designer, a list of genes of interest for a fluorescence in-situ hybridization experiment, the genes of interest associated with a transcriptome;
construct a library of oligonucleotides configured to bind to at least some of the genes of interest, the constructing comprising:
compute possible target regions of the transcriptome,
access a set of probe creation parameters and assigning values to the probe creation parameters;
select possible oligonucleotides for the library based on the possible target regions and the values for the probe creation parameters,
adjust the values for the probe creation parameters, and
automatically iterate over the selecting and adjusting until a stopping condition is met; and
store the constructed library in a non-transitory computer-readable storage medium.

10. The computer-readable storage medium of claim 9, wherein the automatically iterating excludes re-computing the possible target regions of the transcriptome.

11. The computer-readable storage medium of claim 9, wherein the probe creation parameters include one or more of a number of probes, a probe length, or a probe specificity.

12. The computer-readable storage medium of claim 9, wherein the instructions further configure the computer to generate the list of genes of interest by:
receive a gene name for one of the genes of interest;
access a database that maps common gene names to transcript identifiers;
look up the gene name in the database to find a matching transcript identifier for the gene name; and
provide the transcript identifier as part of the list of genes of interest.

13. The computer-readable storage medium of claim 12, wherein the instructions further configure the computer to recognize that the received gene name gene maps to multiple possible transcript identifiers, and offering the multiple possible transcript identifiers as gene synonyms for selection.

14. The computer-readable storage medium of claim 12, wherein each of the transcript identifiers in the database is associated with version information, and the version information is omitted from the list of genes of interest provided as input to the probe designer.

15. The computer-readable storage medium of claim 9, wherein data accessed by the probe designer is managed by a back end server and computations performed by the probe designer are performed on a front end server distinct from the back end server.

16. The computer-readable storage medium of claim 9, construct the library is performed by instructions that are written in a non-script-based language.

17. A computing apparatus comprising:
a processor; and
a memory storing instructions that, when executed by the processor, configure the apparatus to:
receive, as an input at a probe designer, a list of genes of interest for a fluorescence in-situ hybridization experiment, the genes of interest associated with a transcriptome;
construct a library of oligonucleotides configured to bind to at least some of the genes of interest, the constructing comprising:
compute possible target regions of the transcriptome,
access a set of probe creation parameters and assigning values to the probe creation parameters;
select possible oligonucleotides for the library based on the possible target regions and the values for the probe creation parameters,
adjust the values for the probe creation parameters, and
automatically iterate over the selecting and adjusting until a stopping condition is met; and
store the constructed library in a non-transitory computer-readable storage medium.

18. The computing apparatus of claim 17, wherein the automatically iterating excludes re-computing the possible target regions of the transcriptome.

19. The computing apparatus of claim 17, wherein the probe creation parameters include one or more of a number of probes, a probe length, or a probe specificity.

20. The computing apparatus of claim 17, wherein the instructions further configure the apparatus to generate the list of genes of interest by:
receive a gene name for one of the genes of interest;
access a database that maps common gene names to transcript identifiers;
look up the gene name in the database to find a matching transcript identifier for the gene name; and
provide the transcript identifier as part of the list of genes of interest.

21. The computing apparatus of claim 20, wherein the instructions further configure the apparatus to recognize that the received gene name gene maps to multiple possible transcript identifiers, and offering the multiple possible transcript identifiers as gene synonyms for selection.

22. The computing apparatus of claim 20, wherein each of the transcript identifiers in the database is associated with version information, and the version information is omitted from the list of genes of interest provided as input to the probe designer.

23. The computing apparatus of claim 17, wherein data accessed by the probe designer is managed by a back end server and computations performed by the probe designer are performed on a front end server distinct from the back end server.

24. The computing apparatus of claim 17, construct the library is performed by instructions that are written in a non-script-based language.
